(19) United States
(12) Patent Application Publication
(10) Pub. No.: US 2025/0368503 A1
(43) Pub. Date: Dec. 4, 2025

(54) PHOTOCATALYTIC PANEL AND METHODS FOR CONTINUOUS HYDROGEN PRODUCTION

(71) Applicants: QDSol, LTD., Rehovot (IL); TECHNION RESEARCH AND DEVELOPMENT FOUNDATION, LTD., Haifa (IL)

(72) Inventors: Lilac Amirav, Zichron Yaakov (IL); Lior Yedidya, Jerusalem (IL)

(21) Appl. No.: 18/875,378

(22) PCT Filed: Jun. 16, 2023
(86) PCT No.: PCT/IB2023/000363

§ 371 Date: Dec. 16, 2024

Related U.S. Application Data
(60) Provisional application No. 63/353,085, filed on Jun. 17, 2022.

(51) Int. Cl. C01B 3/04; B01J 4/00; B01J 19/12; B01J 35/39; C01B 13/02; C07C 45/42

(52) U.S. Cl. CPC C01B 3/042; B01J 4/001; B01J 19/127; B01J 35/39; C01B 13/0207; C07C 45/42; B01J 2204/002; B01J 2219/00033; B01J 2219/00045; B01J 2219/0869; B01J 2219/0888; B01J 2219/1203

(57) ABSTRACT

The disclosure relates to systems and methods for continuous hydrogen production using photocatalysis. Specifically, the disclosure relates to systems and methods for continuous hydrogen production using photocatalysis of water utilizing semiconductor charge carriers immobilized on removable carriers in the presence of a reducing agent such as tertiary amines.

### BACKGROUND

The disclosure is directed to systems and methods for continuous photocatalytic generation of predetermined products. Specifically, the disclosure is directed to systems and methods for photocatalytic green hydrogen production from water using shaped nanoscale semiconductors, incorporated into a panel via immobilization on certain substrates, in a closed redox cycle.

Photocatalytic panels using sunlight can be used to split water into hydrogen and oxygen, as well as reduce CO₂. However, there are some shortcomings to consider. For example, the panels may not be efficient, while photocatalytic particles in the panels can also degrade over time, which can reduce their (and the panel's) effectiveness. Moreover, to keep them functioning properly, the panels may require regular maintenance. Likewise, and because they rely on sunlight to work, they may not be as effective at splitting water, or purifying waste streams on cloudy days or under precipitation.

Currently, green hydrogen is a primary candidate energy carrier for applications that require a high energy density, or are remote from electricity grids, and it can serve as feedstock for chemical reactions to produce a range of synthetic fuels. It will holed significant rule if insuring future decarbonization of the energy system, and transition to sustainable future. Although industrial methods exist for the production of green hydrogen, those methods are not economical/cost-competitive against fossil-based polluting “Gray” H₂.

One potential technology for producing green hydrogen employs artificial photosynthesis (photocatalysis) for solar driven production of hydrogen from water in a closed redox cycle. In such photocatalytic processes, light induced electron-hole pairs generated in a semiconductor particle are utilized for the promotion of the desired chemical reactions with various substances. Nanostructuring and shaping the photocatalytic particle system were found to greatly improve the system's overall efficiency.

Given that photocatalytic semiconductor nanoparticles with sufficient efficiency and stability for solar green hydrogen production are only now starting to emerge, systems and methods for their incorporation into a panel that enables continuous operation were not yet developed.

Stable semiconductor system with sufficient (although narrow) band gap absorption (e.g., 2.4>X>2.0 eV) in the visible range, and electron affinity for visible light absorption and for driving the subsequent redox chemistry, are challenging. Additional challenges facing the photocatalytic process is the inevitable recombination of photoinduced charge carriers, particle disintegration, back reaction of intermediates on the catalyst surface, and the back reaction of the products, in other words, non-desirable termination reactions.

The disclosed systems and methods intend to address these deficiencies.

### SUMMARY

Disclosed, in various exemplary implementations, are systems and methods for water photocatalysis using shaped semiconductor (or photo-induced charge carriers) immobilized on optionally removable transparent membranes under a closed redox cycle.

In an exemplary implementation provided herein is a system for continuously generating hydrogen comprising at least partially transparent container having a first and a second inlets, and a first and a second outlet; a pressurized water source in continuous liquid communication with the first inlet; a pressurized source of an electron donor compound; and at least one removable transparent membrane comprising a plurality of at least partially embedded shaped nanoscale semiconductors, each having a basal end and an apical end, with a seed embedded within the shaped nanoscale semiconductors at the basal end, and a metal tip disposed at the apical end of the shaped nanoscale semiconductors.

In yet another exemplary implementation, provided herein is a method of continuously producing hydrogen, implemented in a system comprising a transparent container having a first and a second inlets, and a first and a second outlet; a pressurized water source in liquid communication with the first inlet; a pressurized source of benzylamine (BnNH₂) in liquid communication with the second inlet; and at least one removable transparent membrane comprising a plurality of at least partially embedded shaped nanoscale semiconductors, each having a basal end and an apical end, with a seed embedded within the shaped nanoscale semiconductors at the basal end, and a metal tip disposed at the apical end of the shaped nanoscale semiconductors, the method comprising: using the first inlet, filling the transparent container with water; exposing the transparent container to actinic radiation at a predetermined wavelength; using the plurality of shaped nanoscale semiconductors, photocatalyzing the water to form hydrogen, oxygen, and depleted water; using the second inlet, contacting the container in the presence of a nitrogen source, with accumulated benzylaldehyde; using the first outlet, collecting the hydrogen; and using the second outlet, removing the depleted water.

In even yet another exemplary implementation, provided herein is a method of continuously producing benzylamine, implemented in a system comprising a transparent container having a first and a second inlets, and a first and a second outlet; a pressurized water source in liquid communication with the first inlet; a pressurized source of benzylamine (BnNH₂) in liquid communication with the second inlet; and at least one removable transparent membrane comprising a plurality of at least partially embedded shaped nanoscale semiconductors, each having a basal end and an apical end, with a seed embedded within each of the shaped nanoscale semiconductors at the basal end, and a metal tip disposed at the apical end of each of the shaped nanoscale semiconductors, the method comprising: using the first inlet, filling the transparent container with water; exposing the transparent container to actinic radiation at a predetermined wavelength; using the plurality of shaped nanoscale semiconductors, photocatalyzing the water to form hydrogen, oxygen, and depleted water; using the second inlet, contacting the container in the presence of a nitrogen source (e.g., NH₃ at a predetermined molar concentration), with the benzylamine; using the first outlet, collecting the hydrogen; using the second outlet, removing the depleted water; and separating accumulated benzyladehyde from the depleted water (e.g., by evaporation).

These and other features of the systems and methods for water photocatalysis using nanoscale shaped semiconductors immobilized on removable membranes will become apparent from the following detailed description when read in conjunction with the figures and examples, which are exemplary, not limiting.

### BRIEF DESCRIPTION OF THE DRAWINGS

For a better understanding of the systems and methods for using water photocatalysis using semiconductor rods immobilized on removable membranes in the presence of a reducing agent, with regard to the exemplary implementations thereof, reference is made to the accompanying examples and figures, in which:

FIG. 1, illustrates a schematic of an exemplary implementation of the system;

FIG. 2, is a schematic showing an exemplary implementation of the semiconductor rods; and

FIG. 3, is a schematic showing an exemplary photocatalytic closed cycle redox reaction.

### DETAILED DESCRIPTION

Provided herein, are exemplary implementations of systems and methods for continuous hydrogen production through photocatalysis of water utilizing semiconductor charge carriers immobilized on removable transparent membranes, optionally in the presence of a reducing agent, as well as the water.

As distinct from bulk photocatalysts, realized as thin films on conducting substrates, water splitting with nanoscale photocatalysts simply utilizes a photocatalyst material immersed in water. The principles of photocatalytic water splitting require high surface areas for electron excitation and collection, and the use of oriented nanocatalysts, which offer high surface to volume ratios (A/V) and high light harvesting efficiencies. The disclosed semiconductor nanostructures improve photocatalysis through the combined effects of quantum confinement and unique surface morphologies.

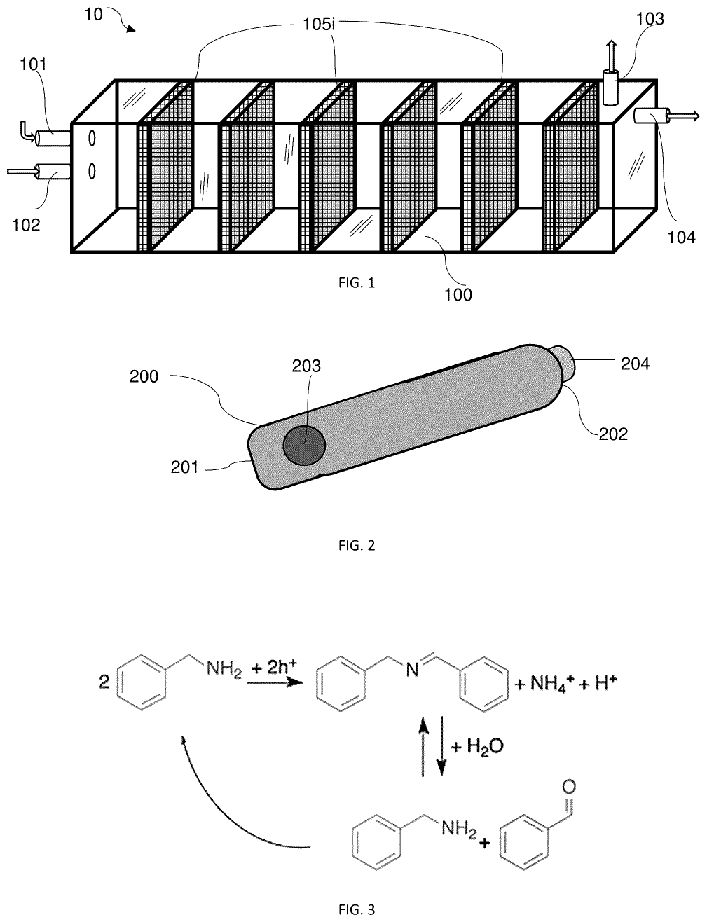

Accordingly and in an exemplary implementation as disclosed in FIGS. 1-2, provided herein is system 10 for continuously generating hydrogen comprising transparent container 100 having first 101 and second 102 inlets, and first 103 and second 104 outlet; pressurized water source 111 in continuous (in other words, continuous flow) liquid communication with first inlet 101; pressurized source of benzylamine 112 in liquid communication with second inlet 102; and at least one i^{th} removable transparent membrane 105i comprising plurality of at least partially embedded shaped nanoscale semiconductors 200j (see e.g., rods, FIG. 2), each semiconductor rod 205, having basal end 201 and apical end 202, with seed 203 embedded within nanoscale j^{th} semiconductor rod 200j at basal end 201, and metal tip 204 disposed at apical end 203 of nanoscale j^{th} semiconductor rod 200j. Metal tip 204, is a co-catalysts loaded on apical end 202 of semiconductor rod 205, operable as a photocatalysts that can act as reaction site and catalyze the progress of the reaction, promoting charge separation and migration driven by the interfacial junction formed between the cocatalyst and the semiconductor rod. The co-catalyst can be, for example, any metal with a relatively large work function (Φm>4.5 eV), such as, for example, platinum (Pt), palladium (Pd) Rhodium (Rh), Cobalt (Co), Nickel (Ni), Copper (Cu) and the like.

In certain exemplary implementations, the shaped nanoscale semiconductors can be any suitable shape that will allow charge separation between seed 203 and metal tip 204. These shapes can be, for example, at least one of: rods, platelets, spheres, cubes, and core/shell semiconductors.

The removable transparent membrane used in the systems disclosed and the methods implemented in these systems, is a transparent mesh filter sized and configured to increase the surface area exposing the plurality of shaped nanoscale semiconductors 200j. The mesh can be a mesh sieve having a nominal sieve opening of between about 210 μM (size 70), and about 5,000 μM and will depend on the size and spatial configuration of shaped nanoscale semiconductors 200j.

Additionally, shaped nanoscale semiconductors 200j can be directly adsorbed onto particles, such as silica particles and suspended within the continuously flowing mixture of water and for example, benzaldehyde. As illustrated in FIG. 1, filter 106 can be disposed downstream from first outlet 103, as well as second outlet 104, and be sized and configured to prevent the flow of the carrier particles 2000p (not shown). Carrier particles 2000p, can be silica particles, or glass beads (about 450 μm) having an average volume average diameter (D_{3,2}) of between about 10 μm and about 500 μm, as measured by, for example, laser scattering. The particles can be, for example, adsorbed onto the glass beads, or be partially embedded within the pores of the silica beads (interchangeable with ‘particles’). The porous particles can have an average pore diameter of between about 0.5 nm (5 angstrom A) and about 50 nm, as measured by, for example, Helium pycnometry.

In the context of the disclosure, the term “transparent”, or “partially transparent”, refers to a wall or any other composition capable of at least 70% transmission of light. The light referred to can be, e.g., sunlight (filtered or not), actinic light (e.g., from a laser), emitted light (e.g., from a fluorochrome), light of a given wavelength range, or a combination of the foregoing, or transmittance of at least 80%, for example at least 85%, or at least 90%, as measured spectrophotometrically using water as a standard (100% transmittance) at 690 nm. The term “transparent” as used herein would also refer to a composition that transmits at least 70% in the region ranging from 330 nm to 800 nm with a haze of less than 10%. Likewise, the term “wall” which can be interchangeable with the term “aspect”, can be used throughout to identify the various layers regardless of thickness and be rigid made of thermoplastic material, silicone glass or other glassy and/or crystalline state minerals and polymers.

In an exemplary implementation, a predetermined amount of semiconductor rod 200j, and a predetermined amount of a thermoplastic polymer, copolymer, terpolymer and their combination; such as, for example, poly(acrylonitrile) (PAN), poly(carbonate) (PC), poly(siloxane) (PS), poly(dimethylsiloxane) (PDMS), their copolymers, terpolymers and/or combination are suspended and dissolved respectively in a predetermined amount of an organic solvent (e.g., dimethyl furfural (DMF), acetone, diisopropylamine, triethylamine, pentane, and xylenes). The suspension is then stirred for a given period to make sure that all the thermoplastic polymer, copolymer, terpolymer and their combination are dissolved and that the semiconductor rods 200j are well dispersed to form a homogenous suspension. The suspension can then be cast (e.g., on glass) for example (or spun/extruded in other examples), and the solvent removed (or woven if spun, cooled if extruded) to form the sieve. Moreover, the loading of shaped nanoscale semiconductors 200j in the thermoplastic polymer, copolymer, terpolymer and/or their combination is configured in an exemplary implementation to provide an optimal conversion.

Accordingly and in another exemplary implementation, provided herein is transparent membrane 105i, each i^{th} removable transparent membrane 105i comprising plurality of at least partially embedded shaped nanoscale semiconductors 200j, each j^{th} semiconductor rod 200j having basal end 201 and apical end 202, with seed 203 embedded within each j^{th} shaped nanoscale semiconductor 200j at basal end 201, and metal tip 204 disposed at apical end 202 of the j^{th} shaped nanoscale semiconductor 200j.

As illustrated in FIG. 2, each j^{th} shaped nanoscale semiconductors 200j, if is a rod, defines a longitudinal axis XL, and can have length ‘L parallel to longitudinal axis XL, of between about 25 nm and about 75 nm, adapted based on the composition of each j^{th} semiconductor rod 200j to separate the charge (electron to metal tip 204, with hole to seed 203). Hence, in an exemplary implementation, seed 203 can be a first Cadmium chalcogenide (e.g., Cadmium selenide (CdSe), and Cadmium telluride (CdTe)), and each j^{th} semiconductor rod 200j is formed of at least one of: second Cadmium chalcogenide (e.g., Cadmium sulfide (CdS), and Cadmium telluride (CdTe)), and titanium dioxide (TiO₂).

Furthermore, in the context of the disclosure, the term “semiconductor” refers to a material with electrical conductivity intermediate in magnitude between that of a conductor and an insulator. The semiconductor may be an intrinsic semiconductor, an n-type semiconductor or a p-type semiconductor. Examples of semiconductors include perovskites; oxides of titanium, niobium, tin, zinc, cadmium, copper or lead; chalcogenides of antimony, copper, zinc, iron, or bismuth (e.g. copper sulphide and iron sulphide); copper zinc tin chalcogenides, for example, copper zinc tin sulphides such a Cu₂ZnSnS₄(CZTS) and copper zinc tin sulphur-selenides such as Cu₂ZnSn(S₁₋ₓSeₓ)₄ (CZTSSe); copper indium chalcogenides such as copper indium selenide (CIS); copper indium gallium chalcogenides such as copper indium gallium selenides (CuIn₁₋ₓGaₓSe₂) (CIGS); and copper indium gallium diselenide. Further examples are group IV compound semiconductors (e.g. silicon carbide); group III-V semiconductors (e.g. gallium arsenide); group II-VI semiconductors (e.g. cadmium selenide); group I-VII semiconductors (e.g. cuprous chloride); group IV-VI semiconductors (e.g. lead selenide); group V-VI semiconductors (e.g. bismuth telluride); and group II-V semiconductors (e.g. cadmium arsenide); ternary or quaternary semiconductors (eg. Copper Indium Selenide, Copper indium gallium di-selenide, copper zinc tin sulphide, or copper zinc tin sulphide selenide (CZTSSe).

In an exemplary implementation each j^{th} shaped nanoscale semiconductor (e.g., rod) 200j is formed of a hybrid CdS/MO, where MO is a metal oxide, such as: Bi₂O₃ (for example the α-type lattice), In₂O₃, ZnO, and SnO₂, TiO₂ and the like. In metal oxide semiconductors, intrinsic point defects (referring to lattice defects of zero dimensionality of impurity atoms in a pure metal, vacancies and self-interstitials), serving as the donors or acceptors. However, in many cases, the band gaps of metal oxides are wide and the defect levels are too deep to provide high concentration carriers. To wit, albeit photosensitive, and relatively nontoxic, a major TiO₂ deficiency is its wide band gap (3.2 eV) making it active only under UV light which is <5% of the total solar radiation spectrum. Conversely, cadmium sulphide (CdS) absorbs in the visible region, with a narrow direct band gap of 2.4 eV, but has been known to leach Cd²⁺ ions in solution, which are toxic and reduce the quantum efficiency. Accordingly, it is submitted that a hybrid CdS/TiO₂ semiconductor rod 200j having a seed of at least one of Cadmium selenide (CdSe), and Cadmium telluride (CdTe), will have reduced sloughing of Cd²⁺ ions, and increase the mean time between membrane replacements.

Hybrid semiconductor/Metal Oxide complexes are designed in an exemplary implementation on the molecular scale, which allows precise control of functionality, including photophysical, electrochemical, and catalytic properties. For example, using p (orbital)-type metal sulfides and CoOₓ-loaded BiVO₄ working as reduction and oxidation photocatalysts respectively, reduction under visible light can be achieved, with water as the electron donor.

Turning back to FIG. 1, system 10 hydrogen container 150, in liquid communication with first outlet 103; and benzyl amine container 160, in liquid communication with second outlet 104.

In certain exemplary implementations, the methods disclosed are implemented in the systems disclosed. Accordingly, provided herein is a method of continuously producing hydrogen, implemented in a system comprising a transparent container having a first and a second inlets, and a first and a second outlet; a pressurized water source in liquid communication with the first inlet; a pressurized source of benzylamine in liquid communication with the second inlet; and at least one removable transparent membrane comprising a plurality of at least partially embedded shaped nanoscale semiconductors, each having a basal end and an apical end, with a seed embedded within the shaped nanoscale semiconductors at the basal end, and a metal tip disposed at the apical end of the shaped nanoscale semiconductors, the method comprising: using the first inlet, filling the transparent container with water; exposing the transparent container to at least one of: sunlight (filtered or not), actinic light (e.g., from a laser), emitted light (e.g., from a fluorochrome), light of a given wavelength range, and a combination of the foregoing; using the plurality of shaped nanoscale semiconductors, photocatalyzing the water to form hydrogen, oxygen, and depleted water; using the second inlet, contacting the container in the presence of a nitrogen source, with the benzylaldehyde; using the first outlet, collecting the hydrogen; and using the second outlet, removing the depleted water. Then periodically, replacing at least one removable transparent membrane 105i upon determination that conversion efficiency n has decreased, in other words, when the half reaction has a conversion efficiency of less than, for example, 61% measured as described in Equation 1:

$$η = \frac{{r}_{H 2} \cdot Δ G}{P \cdot A} \qquad ( EQU . 1 )$$

where the r_{H2} is the rate of hydrogen generation in mol/s, ΔG is the Gibbs free energy change for the generation of one mole of H₂ by splitting water under standard conditions (237.2 KJ/mol), P is the power density of illumination intensity in W/m² and A is the illuminated area in m².

Similarly, and as illustrated in FIG. 3, the systems disclosed can be used to generate benzaldehyde. As illustrated, the reaction turns benzylamine introduced under pressure, to benzyl aldehyde as follows: first, benzylamine (BnNH₂) is selectively converted into benzylidenebenzylamine after which, the presence of water promotes hydrolysis of the benzylidenebenzylamine to its components, namely benzaldehyde and benzylamine. Continuous irradiation leads to further oxidation of the newly formed BnNH₂, shifting the reaction balance out of equilibrium towards the accumulation of benzaldehyde. The reaction can therefore be used in certain implementations, to remove oxygen from the split water, which can adversely affect the shaped nanoscale semiconductors. Benzylamine can be used in certain implementations, for example, for chemical synthesis, production of pesticides, polymer auxiliaries, and pharmaceutical substances. Accordingly, provided herein is a method of continuously producing benzaldehyde, implemented in a system comprising a transparent container having a first and a second inlets, and a first and a second outlet; a pressurized water source in liquid communication with the first inlet; a pressurized source of benzylamine in liquid communication with the second inlet; and at least one removable transparent membrane comprising a plurality of at least partially embedded shaped nanoscale semiconductors, each having a basal end and an apical end, with a seed embedded within the shaped nanoscale semiconductors at the basal end, and a metal tip disposed at the apical end of the shaped nanoscale semiconductors, the method comprising: using the first inlet, filling the transparent container with water; (continuously) exposing the transparent container to at least one of: sunlight, actinic light, emitted light, light of a given wavelength range, and a combination of the foregoing; using the plurality of shaped nanoscale semiconductors, photocatalyzing the water to form hydrogen, oxygen, and depleted water; using the second inlet, contacting the container in the presence of a nitrogen source, with the benzylamine (BnNH₂) using the first outlet, collecting the hydrogen; using the second outlet, removing the depleted water; and separating accumulated benzyladehyde from the depleted water.

In an exemplary implementation, the hydrogen collected from the first outlet is further purified and compressed to a predetermined pressure (e.g., between about 150 psi and 500 psi) using a gas processing module, included with the system. The gas-processing module, which can comprise compressor, separator, and purifier, is used in another example, to condition the hydrogen. for example, using heat exchanger/condenser included with the gas-processing module, the gas stream is cooled to a predetermined temperature, (e.g., between about 24° C. and about 45° C.) thereby removing water vapor and reducing flow volume to the compressor. For example, a triplex pump compressor with intercooling is selected in an exemplary implementation for the compressor. In addition, the systems can further comprise a plurality of sensors, such as thermocouples, oxygen sensors, pressure sensors, flow meters and the like and be operably coupled to a central processing module, operable to control the various method steps.

The term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

All ranges disclosed herein are inclusive of the endpoints, and the endpoints are independently combinable with each other. “Combination” is inclusive of blends, mixtures, alloys, reaction products, and the like. The terms “a”, “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the rod(s) includes one or more rod). Reference throughout the specification to “one exemplary implementation”, “another exemplary implementation”, “an exemplary implementation”, and so forth, when present, means that a particular element (e.g., feature, structure, and/or characteristic) described in connection with the exemplary implementation is included in at least one exemplary implementation described herein, and may or may not be present in other exemplary implementations. In addition, it is to be understood that the described elements may be combined in any suitable manner in the various exemplary implementations.

In the context of the disclosure, the term “operable” means the system and/or the device and/or the program, or a certain element or step is fully functional, sized, adapted and calibrated, comprises elements for, and meets applicable operability requirements to perform a recited function when activated, coupled, implemented, actuated, effected, realized, or when an executable program is executed by at least one processor associated with the system and/or the device. In relation to systems and circuits, the term “operable” means the system and/or the circuit is fully functional and calibrated, comprises logic for, having the hardware and firmware necessary, as well as the circuitry for, and meets applicable operability requirements to perform a recited function when executed by at least one processor.

All ranges disclosed herein are inclusive of the endpoints, and the endpoints are independently combinable with each other. Furthermore, the terms “first,” “second,” and the like, herein do not denote any order, quantity, or importance, but rather are used to denote one element from another.

Likewise, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not stated as such.

Accordingly, provided herein is a system for continuously generating solar driven hydrogen comprising a partially transparent container having inlet(s), and an outlet; a pressurized water source in continuous liquid communication with an inlet; a pressurized source of an electron donor in liquid communication with inlet; and a membrane or substrate comprising a plurality of at least partially embedded or anchored shaped nanoscale semiconductor particles, wherein (i) the membrane is comprised of beads (e.g., silica, glass, thermoplastic), (ii) the membrane is partially or fully removable, wherein (iii) the embedded or anchored shaped nanoscale semiconductor particles are removable (in other words, independently from the carriers/substrate/membrane), wherein (iv) the membrane is operable as a light sensitizer, as a chemically stabilizer (referring to the material's ability of performing photoinduced charge generation, photoemission or electroemission), or as an enhancer of the stability of the shaped nanoscale semiconductor particles, (v) the shaped nanoscale semiconductor particles comprise at least two different semiconductors, with a band alignment that supports a closed redox cycle, and (vi) an additional co-catalyst domain, operable to affect charge separation, serving as catalytic site, lowering the activation potential for a redox half reaction, wherein (vii) the shape (of the shaped nanoscale semiconductor particles), is at least one of: a rod, a wire, a platelet, a sheet, a spheres, a cube, a tetrapod, a multipod, and a core-shell semiconductor shape, wherein (viii) the shaped nanoscale semiconductor size is between about 2 nanometer (nm) and about 100 nm, wherein (ix) at least one shaped nanoscale semiconductor has suitable band gap and electron affinity to support visible light production of hydrogen from water, and (x) is Cadmium chalcogenide, wherein (xi) the co-catalyst is: nickel, platinum, bimetallic co-catalyst, or a transition metal chalcogenides, (xii) the first Cadmium chalcogenide is at least one of Cadmium selenide (CdSe), and Cadmium sulfide (CdS), and wherein the system further comprising (xiii) a hydrogen container, in communication with the an outlet; and a container for the oxidized electron donor, in communication with an outlet.

In another exemplary implementation, provided herein is a method of continuously producing hydrogen, implemented in a system comprising a transparent container having a first and a second inlets, and a first and a second outlet; a pressurized water source in continuous liquid communication with the first inlet; a pressurized source of benzylamine in liquid communication with the second inlet; and at least one removable transparent membrane comprising a plurality of at least partially embedded shaped nanoscale semiconductors, each shaped nanoscale semiconductor having a basal end and an apical end, with a seed embedded within each of the shaped nanoscale semiconductors at the basal end, and a metal tip disposed at the apical end of each of the shaped nanoscale semiconductors, the method comprising: using the first inlet, continuously (in a continuous flow) filling the transparent container with water; exposing the transparent container to at least one of: sunlight, actinic light, emitted light, light of a given wavelength range, and a combination of the foregoing; using the plurality of shaped nanoscale semiconductors, photocatalyzing the water to produce hydrogen, oxygen, and depleted water; using the second inlet, contacting the container in the presence of a nitrogen source, with the benzylamine (BnNH₂); using the first outlet, collecting the hydrogen; and using the second outlet, continuously removing the depleted water, the method further comprising (xiv) periodically removing at least one removable transparent membrane, or beads; and replacing the removable transparent membrane or beads with an unexposed removable transparent membrane or unexposed beads having the shaped adsorbed, or partially embedded shaped nanoscale semiconductor(s) coupled thereto.

In yet another exemplary implementation, provided herein is a method of continuously producing benzaldehyde, implemented in a system comprising a transparent container having a first and a second inlets, and a first and a second outlets; a pressurized water source in liquid communication with the first inlet; a pressurized source of benzylamine (BnNH₂) in liquid communication with the second inlet; and at least one removable transparent membrane, or a plurality of beads, each comprising a plurality of at least partially embedded shaped nanoscale semiconductors, each shaped nanoscale semiconductor having a basal end and an apical end, with a seed embedded within each shaped nanoscale semiconductor at the basal end, and a metal tip disposed at the apical end of each shaped nanoscale semiconductor, the method comprising: using the first inlet, continuously filling the transparent container with water; exposing the transparent container to at least one of: sunlight, actinic light, emitted light, light of a given wavelength range, and a combination of the foregoing; using the plurality of shaped nanoscale semiconductors, photocatalyzing the water to produce hydrogen, oxygen, and depleted water; using the second inlet, contacting the container in the presence of a nitrogen source, with the benzylamine (BnNH₂); using the first outlet, collecting the hydrogen; using the second outlet, removing the depleted water; and separating the accumulated benzaldehyde from the depleted water, the method further comprising (xv) periodically (e.g., when determined appropriate, for example upon detecting a drop in hydrogen production of more than 5%, or 10% or 20% or 30% from the initial hydrogen production), removing at least one removable transparent membrane, or at least a portion of the plurality of beads; and replacing the removable transparent membrane, or the portion of the plurality of beads with an unexposed removable transparent membrane, or a portion of unexposed plurality of beads having the shaped adsorbed, or partially embedded shaped nanoscale semiconductor(s) coupled thereto.

The above examples and description have of course been provided only for the purpose of illustration, and are not intended to limit the disclosed technology in any way. As will be appreciated by the skilled person, the disclosed technology can be carried out in a great variety of ways, employing more than one technique from those described above, all without exceeding the scope of the invention.

1. A system for continuously generating solar driven hydrogen comprising
a) a partially transparent container having inlet(s), and an outlet;
b) a pressurized water source in continuous liquid communication with an inlet;
c) a pressurized source of an electron donor in liquid communication with inlet; and
d) a membrane or substrate comprising a plurality of at least partially embedded or anchored shaped nanoscale semiconductor particles.

2. The system of claim 1, wherein the membrane is a transparent mesh filter.

3. The system of claim 1, wherein the membrane is comprised of beads.

4. The system of claim 3, wherein the membrane is partially or fully removable.

5. The system of claim 1, wherein the embedded or anchored shaped nanoscale semiconductor particles are removable.

6. The system of claim 1, wherein the membrane is operable as a light sensitizer, as a chemically stabilizer, or as an enhancer of the stability of the shaped nanoscale semiconductor particles.

7. The system of claim 1, wherein the shaped nanoscale semiconductor particles comprise at least two different semiconductors, with a band alignment that supports a closed redox cycle.

8. The system of claim 1, wherein the shaped nanoscale semiconductor particles comprise an additional cocatalyst domain, operable to affect charge separation, serving as catalytic site, lowering the activation potential for a redox half reaction.

9. The system of claim 8, wherein the shape is at least one of: a rod, a wire, a platelet, a sheet, a spheres, a cube, a tetrapod, a multipod, and a core/shell semiconductor.

10. The system of claim 8, wherein the shaped nanoscale semiconductor size is between about 2 nanometer (nm) and about 100 nm.

11. The system of claim 7, wherein at least one shaped nanoscale semiconductor has suitable band gap and electron affinity to support visible light production of hydrogen from water.

12. The system of claim 11, wherein at least one shaped nanoscale semiconductor is Cadmium chalcogenide.

13. The system of claim 8, wherein the cocatalyst is: nickel, platinum, bimetallic cocatalyst, or a transition metal chalcogenides.

14. The system of claim 12, wherein the first Cadmium chalcogenide is at least one of Cadmium selenide (CdSe), and Cadmium sulfide (CdS).

15. The system of claim 1, further comprising:
a) a hydrogen container, in communication with the an outlet; and
b) a container for the oxidized electron donor, in communication with an outlet.

16. A method of continuously producing hydrogen, implemented in a system comprising a transparent container having a first and a second inlets, and a first and a second outlet; a pressurized water source in continuous liquid communication with the first inlet; a pressurized source of benzylamine in liquid communication with the second inlet; and at least one removable transparent membrane comprising a plurality of at least partially embedded shaped nanoscale semiconductors, each shaped nanoscale semiconductor having a basal end and an apical end, with a seed embedded within each of the shaped nanoscale semiconductors at the basal end, and a metal tip disposed at the apical end of each of the shaped nanoscale semiconductors, the method comprising:
a) using the first inlet, continuously filling the transparent container with water;
b) exposing the transparent container to at least one of: sunlight, actinic light, emitted light, light of a given wavelength range, and a combination of the foregoing;
c) using the plurality of shaped nanoscale semiconductors, photocatalyzing the water to produce hydrogen, oxygen, and depleted water;
d) using the second inlet, contacting the container in the presence of a nitrogen source, with the benzylamine (BnNH₂);
e) using the first outlet, collecting the hydrogen; and
f) using the second outlet, removing the depleted water.

17. The method of claim 16, further comprising periodically removing at least one removable transparent membrane; and replacing the removable transparent membrane with an unexposed removable transparent membrane.

18. A method of continuously producing benzaldehyde, implemented in a system comprising a transparent container having a first and a second inlets, and a first and a second outlets; a pressurized water source in liquid communication with the first inlet; a pressurized source of benzylamine (BnNH₂) in liquid communication with the second inlet; and at least one removable transparent membrane, or a plurality of beads, each comprising a plurality of at least partially embedded shaped nanoscale semiconductors, each shaped nanoscale semiconductor having a basal end and an apical end, with a seed embedded within each shaped nanoscale semiconductor at the basal end, and a metal tip disposed at the apical end of each shaped nanoscale semiconductor, the method comprising:
a) using the first inlet, continuously filling the transparent container with water;
b) exposing the transparent container to at least one of: sunlight, actinic light, emitted light, light of a given wavelength range, and a combination of the foregoing;
c) using the plurality of shaped nanoscale semiconductors, photocatalyzing the water to produce hydrogen, oxygen, and depleted water;
using the second inlet, contacting the container in the presence of a nitrogen source, with the benzylamine (BnNH₂);
e) using the first outlet, collecting the hydrogen;
f) using the second outlet, removing the depleted water; and
g) separating the accumulated benzaldehyde from the depleted water.

19. The method of claim 18, further comprising periodically removing at least one removable transparent membrane, or at least a portion of the plurality of beads; and replacing the removable transparent membrane, or the portion of the plurality of beads with an unexposed removable transparent membrane, or a portion of unexposed plurality of beads having the shaped adsorbed, or partially embedded shaped nanoscale semiconductor(s) coupled thereto.
